(19) United States
(12) Patent Application Publication
(10) Pub. No.: US 2021/0290431 A1
(43) Pub. Date: Sep. 23, 2021

(54) DEVICES, SYSTEMS, AND METHODS FOR CONTROLLING TEMPERATURE IN PEDIATRIC SUBJECTS

(71) Applicant: Advanced Cooling Therapy, Inc., Chicago, IL (US)

(72) Inventors: Erik Kulstad, Chicago, IL (US); Patrick Shanley, Chicago, IL (US)

(21) Appl. No.: 17/339,514

(22) Filed: Jun. 4, 2021

Related U.S. Application Data
(63) Continuation of application No. 16/041,183, filed on Jul. 20, 2018.
Application No. 16/041,183 is a continuation of application No. 14/017,784, filed on Sep. 4, 2013, now abandoned.
(60) Provisional application No. 61/697,112, filed on Sep. 5, 2012.

(51) Int. Cl. A61F 7/12

(52) U.S. Cl. CPC A61F 7/12; A61F 2007/0056

(57) ABSTRACT

Methods, devices, and systems for temperature management of a pediatric patient in a perioperative environment are disclosed. Methods, devices, and systems for maintaining normothermia in a pediatric patient in a perioperative environment are disclosed. The use of esophageal heat transfer devices for perioperative temperature management and/or maintenance of normothermia in a pediatric patient is disclosed.

### RELATED APPLICATIONS

This application is a continuation of U.S. patent application Ser. No. 16/041,183, filed on Jul. 20, 2018, which is a continuation of U.S. patent application Ser. No. 14/017,784, filed on Sep. 4, 2013, which claims the benefit of U.S. Provisional Application No. 61/697,112, filed on Sep. 5, 2012. Both applications are incorporated herein by reference in their entirety.

### FEDERALLY SPONSORED RESEARCH OR DEVELOPMENT

This invention was made with Government support under National Science Foundation (NSF) Award Number 1142664. The Government has certain rights in this invention.

### FIELD OF THE INVENTION

The present invention relates to methods, devices, and systems for controlling body temperature in pediatric subjects. In one aspect, the present invention relates to esophageal heat transfer devices and systems for maintenance of body temperature in an anesthetized pediatric subject in an operating room. In another aspect, the present invention relates to esophageal heat transfer devices and systems for inducing therapeutic hypothermia in an anesthetized pediatric subject in an operating room. In still another aspect, the present invention relates to esophageal heat transfer devices and systems for warming or re-warming an anesthetized pediatric subject in an operating room.

### BACKGROUND OF THE INVENTION

In humans, thermoregulatory processes maintain body temperature within narrow limits, usually 36.5-37.5° C. In a surgical setting, however, the normal balance between heat loss and production is often disrupted, leading to inadvertent hypothermia. Inadvertent hypothermia can be the result of increased heat loss due to, for example, exposure of the body surface to a low temperature environment, administration of cold intravenous (IV) fluids, and/or disruption of the thermoregulatory processes by general anesthetic agents. For example, general anesthesia typically leads to hypothermia comprising a rapid reduction of 1.0-1.5° C. in the core temperature.

Inadvertent hypothermia has been demonstrated to adversely impact a wide range of clinical factors. Even inadvertent mild hypothermia (<1° C.) during operative procedures increases the incidence of wound infection, prolongs hospitalization, increases the incidence of morbid cardiac events and ventricular tachycardia, and impairs coagulation. Mild hypothermia significantly increases blood loss by approximately 16% and increases the relative risk for transfusion by approximately 22%, while maintaining perioperative normothermia reduces blood loss and transfusion requirement by clinically important amounts.

Because considerable strong evidence shows that thermal management improves outcomes in a variety of surgical patients, the current American Heart Association-American College of Cardiology 2007 Guidelines on Perioperative Cardiovascular Evaluation and Care for Noncardiac Surgery include a Level 1 recommendation for maintenance of perioperative normothermia.

Moreover, recognizing the numerous complications of perioperative hypothermia, the American Society of Anesthesiologists (ASA) has recently recommended that postoperative temperature become a basis for assessing physician compliance with current guidelines on the prevention of hypothermia.

Although inadvertent operative hypothermia is considered one of the most preventable surgical complications, existing methods to control body temperature are limited in efficacy, such that the incidence of inadvertent operative hypothermia for surgical patients can exceed 50%.

Infant and pediatric subjects are at an even greater risk for perioperative hypothermia. This may be due, at least in part, to a greater surface area to body mass ratio, smaller stores of subcutaneous fat, and poor vasomotor control, making infant and pediatric subjects more susceptible to perioperative hypothermia. Indeed, the ASA and the American Association of Nurse Anesthetists (AANA) have recommended continuously monitoring temperature in pediatric patients receiving general anesthesia.

Control of a pediatric patient's body temperature while undergoing a surgical procedure in the operating room is beneficial, but avoiding hypothermia in the perioperative environment is a challenge. Surgical suite temperature is often maintained between about 20° C. (68° F.) and about 24° C. (75.2° F.). For some specialties, the temperatures can vary from about 16.6° C. (62° F.) to about 20° C. (68° F.). In addition, and as noted above, pediatric subjects are at a greater risk for perioperative hypothermia than adult subjects.

Currently available methods to control body temperature during the perioperative period include both non-invasive and invasive techniques. For example, warmed blankets, forced air heating, warmed IV fluids, and warmed humidified gases have been used to decrease heat loss in pediatric and infant subjects in the perioperative period.

Several issues exist with these current methods: (1) excessively warm room temperature creates an uncomfortable environment for the surgical team, (2) forced-air warmers are bulky and may impact the surgical field; they tend to be inefficient and must be used for extended periods of time in the operating room, and (3) none of these systems adequately control or manage temperature, leading to both overheating or, more often, inadequate warming.

Rasmussen et al. (Forced-air surface warming versus oesophageal heat exchanger in the prevention of perioperative hypothermia. Acta Anaesthesiol Scand. 1998 Mar;42(3):348-52) mention that forced-air warming of the upper part of the body is effective in maintaining normothermia in patients undergoing abdominal surgery of at least 2 h expected duration, while central heating with an esophageal heat exchanger does not suffice to prevent hypothermia. Bräuer et al. (Oesophageal heat exchanger in the prevention of perioperative hypothermia. Acta Anaesthesiol Scand. 1998 Mar;42(10):1232-33) states that an esophageal heat exchanger can only add a small amount of heat to the overall heat balance of the body.

Often the constellation of circumstances in an operating room provides an extremely challenging environment to maintain normothermia, particularly in a smaller subject, such as a pediatric subject. Similarly, the constellation of circumstances in an operating room provides an extremely challenging environment to warm or re-warm a subject, particularly a smaller subject, such as a pediatric subject. In some circumstances, a surgical subject can lose heat at a rate of about 0.03° C. per minute, or about 1.8° C. per hour. Maintaining normothermia or re-warming after hypothermia can be difficult, particularly for a pediatric-sized patient in an extremely heat-hostile environment.

The present invention provides devices, methods, and systems for rapidly and efficiently controlling body temperature of a pediatric subject, while at the same time maintaining access to important anatomical structures. Certain embodiments of the present invention also provide devices, methods, and systems for maintaining normothermia in a pediatric subject during the perioperative period without producing thermoregulatory shivering. Certain embodiments of the present invention also provide devices, methods, and systems for maintaining a pediatric subject's core body temperature within a narrow range with little variation around the goal during the perioperative period. Certain embodiments of the present invention also provide devices, methods, and systems for efficiently re-warming a pediatric subject during the perioperative period.

### BRIEF SUMMARY OF THE INVENTION

An aspect of the present invention provides methods for operative temperature management in a pediatric subject. The methods comprise controlling the subject's body temperature via esophageal cooling or warming. The methods can further comprise positioning part or all of a heat transfer region of a heat transfer device within the subject's esophagus to control the subject's body temperature. In certain embodiments, the heat transfer device comprises a fluid path defined by one or more lumens, which allow for the flow of a heat transfer medium within the device. The methods can further comprise delivering the heat transfer medium to the heat transfer region of the device, via, for example, the fluid path, for a time sufficient to control the subject's body temperature.

Another aspect of the present invention provides methods for maintaining normothermia in a pediatric subject during a perioperative period. The perioperative period can comprise any of the three phases of surgery: preoperative, intraoperative, and postoperative. The methods comprise inserting a heat transfer device into the pediatric subject's esophagus. The device can be inserted through a nostril or mouth of the pediatric subject. The heat transfer device can include a fluid path defined by one or more lumens. In at least one embodiment, the heat transfer device comprises an inflow lumen and an outflow lumen. The methods further comprise initiating flow of a heat transfer medium along the fluid path and circulating the medium along the fluid path for a time sufficient to maintain normothermia in the pediatric subject. The methods can further comprise monitoring one or more physiological parameters of the subject. For example, the subject's body temperature can be monitored using a temperature probe. The temperature probe can be, for example, a gastric temperature probe that is inserted into the subject's stomach via a lumen defined by the heat transfer device. In at least one embodiment, the heat transfer device comprises a gastric access lumen that allows for insertion of a gastric tube or gastric probe.

Yet another aspect of the present invention provides systems and devices for operative temperature management in a pediatric subject. The systems can comprise an esophageal heat transfer device. The esophageal heat transfer device can comprise a heat transfer region that is capable of contacting the esophageal wall of a pediatric subject. In certain embodiments, the esophageal heat transfer device comprises a fluid path defined by one or more lumens, which allows for the flow of the heat transfer medium within the device. The system can further comprise a gastric access lumen and/or a temperature probe. For example, the esophageal heat transfer device can include or define a gastric access lumen. The gastric access lumen can allow for the insertion of a gastric tube or gastric probe. Thus, a separate gastric probe, such as a temperature probe, or separate tube, such as a nasogastric tube or an orogastric tube, can be inserted into the subject via the gastric access lumen. The nasogastric tube, orogastric tube, or gastric probe can be provided with the heat transfer device or provided as an after-market component. Alternatively, the gastric probe or tube can be integrated with the heat transfer device. For example, the heat transfer device can comprise a gastric access tube, which defines the gastric access lumen. In addition to allowing for placement of a separate tube or probe, the gastric access tube of the device can allow for suctioning of gastric contents.

Still another aspect of the present invention provides systems and devices for maintaining normothermia in pediatric subject during a perioperative period. The perioperative period can comprise any of the three phases of surgery: preoperative, intraoperative, and postoperative. The systems can comprise an esophageal heat transfer device. The esophageal heat transfer device can comprise a heat transfer region that is capable of contacting the esophageal wall of a pediatric subject. In certain embodiments, the esophageal heat transfer device comprises a fluid path defined by one or more lumens, which allows for the flow of the heat transfer medium within the device. The system can further comprise a gastric access lumen and/or a temperature probe. For example, the esophageal heat transfer device can include or define a gastric access lumen. The gastric access lumen can allow for the insertion of a gastric tube or gastric probe. Thus, a separate gastric probe, such as a temperature probe, or separate tube, such as a nasogastric tube or an orogastric tube, can be inserted into the subject via the gastric access lumen. The nasogastric tube, orogastric tube, or gastric probe can be provided with the heat transfer device or provided as an after-market component. Alternatively, the gastric probe or tube can be integrated with the heat transfer device. For example, the heat transfer device can comprise a gastric access tube, which defines the gastric access lumen. In addition to allowing for placement of a separate tube or probe, the gastric access tube of the device can allow for suctioning of gastric contents.

Other features, objects, and advantages of the present invention are apparent in the detailed description that follows. It should be understood, however, that the detailed description, while indicating preferred embodiments of the invention, are given by way of illustration only, not limitation. Various changes and modifications within the scope of the invention will become apparent to those skilled in the art from the detailed description.

### BRIEF DESCRIPTION OF THE DRAWINGS

FIG. 1 is a temperature plot depicting temperature modulation achieved with an exemplary heat transfer device according to an embodiment of the present invention.

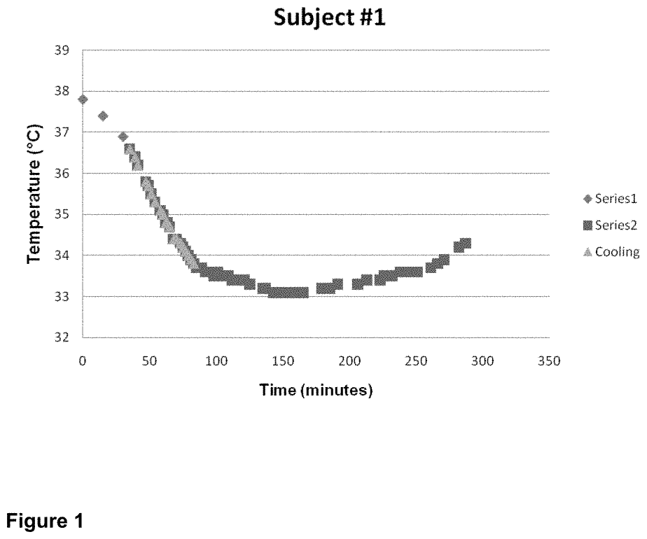

FIG. 2 is a temperature plot depicting temperature modulation achieved with an exemplary heat transfer device according to an embodiment of the present invention.

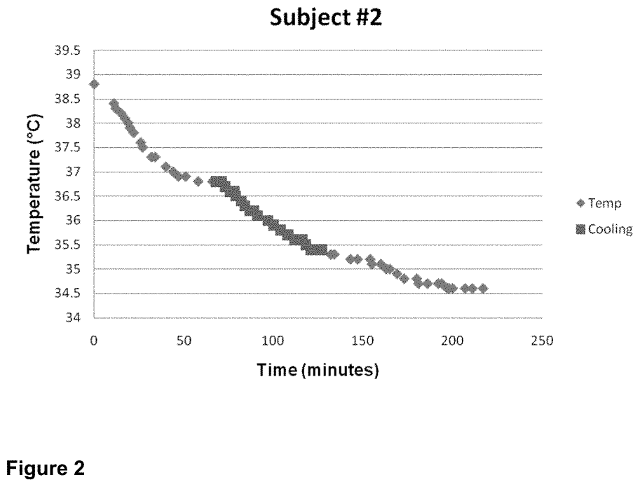

FIG. 3 is a temperature plot depicting temperature modulation achieved with an exemplary heat transfer device according to an embodiment of the present invention.

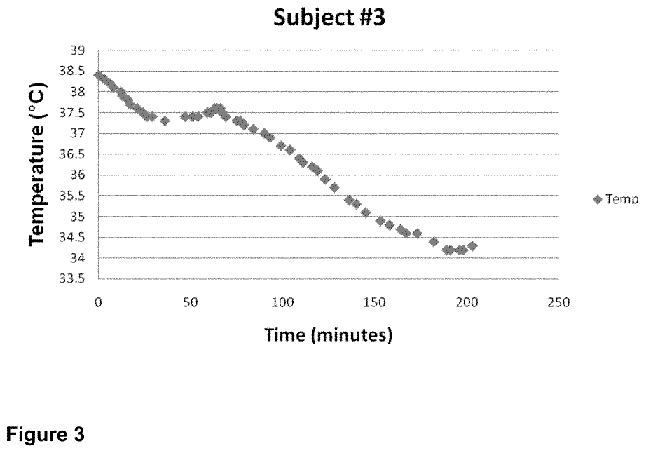

### DETAILED DESCRIPTION OF THE INVENTION

The present methods can include maintaining normothermia in a pediatric subject in a perioperative environment. The methods comprise inserting an esophageal heat transfer device through a nostril or mouth of the pediatric subject and, ultimately, positioning a heat transfer region of the esophageal heat transfer device within the subject's esophagus. The esophageal heat transfer device can comprise an input port for receiving a heat transfer medium from an external source. The input port is connected to a heat transfer medium supply tube, which defines a heat transfer medium supply tube lumen. The esophageal heat transfer device also comprises a heat transfer medium return tube, which defines a heat transfer medium return tube lumen. The heat transfer medium supply tube lumen and the heat transfer medium return tube lumen are in fluid communication with each other and provide a fluid path for the flow of the heat transfer medium. The heat transfer medium supply tube and heat transfer medium return tube are arranged concentrically. In particular, the heat transfer medium return tube is positioned within the heat transfer medium supply tube lumen. The heat transfer medium return tube is connected to an output port for allowing the heat transfer medium to return to the external source.

In certain embodiments, the subject can be less than about 26 kg. In particular, the subject can be between about 21 kg and about 26 kg. In certain embodiments, the subject's skin is left exposed. In certain other embodiments, the subject's skin is covered with a sheet or blanket. In certain embodiments, the ambient temperature of the operating room can be about 21° C. or less. In certain embodiments, the temperature of chilled air being delivered to the operating room from the HVAC system can be about 14° C. or less.

The present methods can include warming or re-warming a pediatric subject in a perioperative environment. The methods comprise inserting an esophageal heat transfer device through a nostril or mouth of the pediatric subject and, ultimately, positioning a heat transfer region of the esophageal heat transfer device within the subject's esophagus. The esophageal heat transfer device can comprise an input port for receiving a heat transfer medium from an external source. The input port is connected to a heat transfer medium supply tube, which defines a heat transfer medium supply tube lumen. The esophageal heat transfer device also comprises a heat transfer medium return tube, which defines a heat transfer medium return tube lumen. The heat transfer medium supply tube lumen and the heat transfer medium return tube lumen are in fluid communication with each other and provide a fluid path for the flow of the heat transfer medium. The heat transfer medium supply tube and heat transfer medium return tube are arranged concentrically. In particular, the heat transfer medium return tube is positioned within the heat transfer medium supply tube lumen. The heat transfer medium return tube is connected to an output port for allowing the heat transfer medium to return to the external source.

For example, a pediatric subject can be re-warmed following a period of hypothermia. The hypothermia can be either inadvertent or induced. For example, a device or system described herein can be used to induce therapeutic hypothermia. Mild therapeutic hypothermia has been shown to improve neurological outcome and decrease mortality in cardiac arrest, hypoxic ischemic encephalopathy, and other conditions in adults and neonates. Following the period of therapeutic hypothermia, the device or system can be used to re-warm the subject.

In certain embodiments, the subject can be less than about 26 kg. In particular, the subject can be between about 21 kg and about 26 kg. In certain embodiments, the subject's skin is left exposed. In certain other embodiments, the subject's skin is covered with a sheet or blanket. In certain embodiments, the ambient temperature of the operating room can be about 21° C. or less. In certain embodiments, the temperature of chilled air being delivered to the operating room from the HVAC system can be about 14° C. or less.

The present methods can include inducing hypothermia in a pediatric subject in a perioperative environment. The methods comprise inserting an esophageal heat transfer device through a nostril or mouth of the pediatric subject and, ultimately, positioning a heat transfer region of the esophageal heat transfer device within the subject's esophagus. The esophageal heat transfer device can comprise an input port for receiving a heat transfer medium from an external source. The input port is connected to a heat transfer medium supply tube, which defines a heat transfer medium supply tube lumen. The esophageal heat transfer device also comprises a heat transfer medium return tube, which defines a heat transfer medium return tube lumen. The heat transfer medium supply tube lumen and the heat transfer medium return tube lumen are in fluid communication with each other and provide a fluid path for the flow of the heat transfer medium. The heat transfer medium supply tube and heat transfer medium return tube are arranged concentrically. In particular, the heat transfer medium return tube is positioned within the heat transfer medium supply tube lumen. The heat transfer medium return tube is connected to an output port for allowing the heat transfer medium to return to the external source.

In certain embodiments, the subject can be less than about 26 kg. In particular, the subject can be between about 21 kg and about 26 kg. In certain embodiments, the subject's skin is left exposed. In certain other embodiments, the subject's skin is covered with a sheet or blanket. In certain embodiments, the ambient temperature of the operating room can be about 21° C. or less. In certain embodiments, the temperature of chilled air being delivered to the operating room from the HVAC system can be about 14° C. or less.

In some embodiments, the subject is an infant. In other embodiments, the subject is a pediatric subject. For example, the subject can be from about 1 year to about 18 years old, including about 1, about 2, about 3, about 4, about 5, about 6, about 7, about 8, about 9, about 10, about 11, about 12, about 13, about 14, about 15, about 16, about 17, or about 18 years old. In some embodiments, the subject is from about 10 kg to about 32 kg. For example, the subject can be about 10, about 11, about 12, about 13,about 14, about 15, about 16, about 17, about 18, about 19, about 20, about 21, about 22, about 23, about 24, about 25, about 26, about 27, about 28, about 29, about 30, about 31 or about 32 kg. In other embodiments, the subject is from about 32 to about 50 kg. For example, the subject can be about 32, about 33, about 34, about 35, about 36, about 37, about 38, about 39, about 40, about 41, about 42, about 43, about 44, about 45, about 46, about 47, about 48, about 49, or about 50 kg.

In certain embodiments, some or all of the subject's skin is exposed during the perioperative period. In certain embodiments, some or all of the subject's skin is covered by a thin sheet or paper during the perioperative period. In certain embodiments, some or all of the subject's skin is covered by a blanket during the perioperative period. In certain embodiments, some or all of the subject's skin is covered by a pre-warmed blanket during the perioperative period.

In some embodiments, the perioperative environment can comprise a surgical suite comprising one or more rooms in which surgical services are provided to a subject. For example, the surgical suite can comprise a room for preparation and anesthesia for the subject, an operating room, and/or a recovery room.

In certain embodiments, the ambient temperature in the surgical suite is less than about 25° C. In particular, the ambient temperature in the operating room is less than about 25° C. In certain embodiments, the ambient temperature in the surgical suite is less than about 23° C. In particular, the ambient temperature in the operating room is less than about 23° C. In certain embodiments, the ambient temperature in the surgical suite is less than about 21° C. In particular, the ambient temperature in the operating room is less than about 21° C. In certain embodiments, the ambient temperature in the surgical suite is less than about 19° C. In particular, the ambient temperature in the operating room is less than about 19° C. In certain embodiments, the ambient temperature in the surgical suite is less than about 17° C. In particular, the ambient temperature in the operating room is less than about 17° C.

In certain embodiments, the surgical suite includes a chilled air supply. In particular, the operating room can include a chilled air supply. The chilled air is delivered through, for example, an HVAC system. The temperature of the chilled air is less than about 21, less than about 19, less than about 17, less than about 15, or less than about 13° C.

In some embodiments, the ambient temperature of the surgical suite is from about 16° C. to about 25° C., including about 16, about 17, about 18, about 19, about 20, about 21, about 22, about 23, about 24, or about 25° C. In some embodiments, the surgical suite includes a chilled air supply. The chilled air can be delivered to the suite through, for example, an HVAC system. In some embodiments, the temperature of the chilled air is from about 12° C. to about 21° C., including about 12, about 13, about 14, about 15, about 16, about 17, about 18, about 19, about 20, or about 21° C.

In some embodiments, the methods described herein employ heat transfer systems and devices such as those described in U.S. Pat. No. 8,231,664; U.S. Patent Publication Nos. 2011/012503, 2011/0125234, and 2011/0130811; and U.S. patent application Ser. No. 13/482,581. The disclosures of each of the aforementioned patents, publications, and applications are hereby incorporated by reference in their entireties.

For example, an esophageal heat transfer device according to an embodiment of the present invention can comprise a heat transfer region that can be placed within a subject's esophagus. The heat transfer region can be, for example, one or more sections of tubing. In certain embodiments, the esophageal heat transfer device can include a heat transfer medium supply tube and a heat transfer medium return tube. The heat transfer medium supply tube and heat transfer medium return tube can be arranged, for example, in parallel or concentrically. In one non-limiting example, the heat transfer medium return tube is positioned within a lumen defined by the heat transfer medium supply tube. In another non-limiting example, the heat transfer medium supply tube is positioned within a lumen defined by the heat transfer medium return tube. In still another non-limiting example, the heat transfer medium supply tube is positioned alongside the heat transfer medium return tube. The lumens of the heat transfer medium supply tube and heat transfer medium return tube can be in fluid communication with each other such that the heat transfer medium can flow along a fluid path defined by the lumens of the heat transfer medium supply tube and heat transfer medium return tube. In certain embodiments, the flow rate of the heat transfer medium along the fluid path can be sufficient to prevent significant fluctuations in the temperature of the heat transfer medium as it flows along the fluid path. For example, the flow rate of the heat transfer medium can be from about 50 mL/min to about 1000 mL/min, preferably about 750 mL/min. As another example, the flow rate of the heat transfer medium can be sufficient to maintain the temperature of the heat transfer medium within about ±3° C., within about ±2° C., within about ±1° C., or within about ±0.5° C. between inlet and outlet temperature. In some embodiments, the flow rate of the heat transfer medium can be sufficient to maintain the temperature of the heat transfer medium within about ±0.1° C. between inlet and outlet temperature.

In certain embodiments, an esophageal heat transfer device can be configured for insertion into a nostril or mouth of a subject. When properly inserted, the heat transfer region of the heat transfer device can be ultimately positioned in the esophagus. Upon placement in the esophagus of a subject, the heat transfer region of the heat transfer device can be in direct contact with the subject's esophagus. In certain embodiments, the heat transfer region of the heat transfer device can contact substantially all of the epithelial surface of a subject's esophagus.

In certain embodiments, an esophageal heat transfer device can comprise one or more ports that allow for ingress and/or egress of the heat transfer medium. For example, the esophageal heat transfer device can include an input port for receiving a heat transfer medium from an external heat exchanger and an output port allowing the heat transfer medium to return to the heat exchanger. In certain embodiments, a supply line is connected to the input port and a return line is connected to the output port. In operation, heat transfer medium enters the input port, flows along a fluid path defined by one or more lumens, and exits the output port. During this process, heat can be transferred from, for example, the heat transfer medium to the esophagus, resulting in an increase and/or stabilization in the temperature of the esophagus, as well as adjacent organs, and ultimately, warming the subject and/or maintaining the subject at normothermia. Alternatively, heat can be transferred from, for example, the esophagus to the heat transfer medium, resulting in a decrease in the temperature of the esophagus, as well as adjacent organs, and ultimately, systemic hypothermia.

In certain embodiments, an esophageal heat transfer device comprises one or more ports connected to one or more lumens, which provide a fluid path for the flow of heat transfer medium, and one or more gastric access tubes. For example, the esophageal heat transfer device can comprise an input port and an output port. The esophageal heat transfer device can further comprise one or more lumens that, physically or functionally, comprise a heat transfer medium supply lumen and a heat transfer medium return lumen. The heat transfer medium supply lumen and the heat transfer medium return lumen can be in fluid communication with each other, thereby defining a fluid path for the flow of the heat transfer medium. The esophageal heat transfer device further comprises one or more gastric access tubes that allows for gastric access when the device is positioned within a subject's esophagus.

In certain embodiments, at least one of the gastric access tubes can allow for gastric suctioning. For example, the proximal end of the gastric tube can be adapted to accommodate attachment to an external suctioning device. The distal portion of the gastric access tube can include one or more ports that provide for communication between the space exterior to the gastric access tube and the gastric access tube lumen. For example, one or more ports can act as a portal between the subject's stomach and the gastric access tube lumen allowing the gastric contents to be suctioned from the subject's stomach out through the gastric access tube lumen. Without wishing to be bound by theory, the ports may improve and enhance the removal of stomach contents, which, in turn, may improve contact between gastric mucosa and the heat transfer device. Such improved contact may enhance heat transfer between the heat transfer device and the gastric mucosa. The presence of multiple ports provides reduced likelihood of blockage of the gastric access tube lumen from semi-solid stomach contents.

All or part of the heat transfer device can be manufactured by, for example, extrusion. Employing such a manufacturing modality eliminates the need to seal junctions or affix end caps and reduce the points at which leaks may occur. Alternatively, or additionally, a fast curing adhesive, such as RTV silicone sealant or temperature-curable sealant can be used to seal junctions and/or bond tubing together. The heat transfer device can be constructed using a biocompatible elastomer and/or plastic, and, optionally, adhesive. For example, biomedical grade extruded silicone rubber such as silicone rubber available from Dow Corning (e.g., Q7-4765, C6-165, and/or C6-550), and an adhesive such as Nusil Med2-4213 can be used to manufacture the heat transfer device.

In certain embodiments, the heat transfer device is capable of cooling at a rate of at least about 3.5° C./hr. In certain embodiments, the heat transfer device is capable of cooling at a rate of about 1.2° C./hr to about 3.5° C./hr. Alternatively, the heat transfer device is capable of cooling at a rate of about 1.2° C./hr to about 1.8° C./hr. In particular, the heat transfer device is capable of cooling at a rate of about 1.8° C./hr.

At least one aspect of the present invention provides a system for controlling core body temperature of a subject, comprising an esophageal heat transfer device insertable within the esophagus of the subject; an external heat exchanger containing a heat transfer fluid; a pump for flowing the heat transfer fluid through a circuit within the esophageal heat transfer device; a heat transfer element in contact with the external heat exchanger; a sensor for detecting a parameter and generating a signal representative of the parameter, wherein the signal is transmitted to a microprocessor to control (i) the flow of heat transfer fluid within the circuit or (ii) the temperature of the heat transfer fluid. The esophageal heat transfer device comprises a heat transfer region, which is configured to contact the subject's esophagus. The sensor can be a temperature sensor configured to generate a signal representing the core body temperature of the subject. The microprocessor can receive a target temperature input and respond to the signal from the temperature sensor via a feedback loop. For example, the feedback loop can employ a proportional integral derivative to control the rate at which the subject approaches the target temperature. The esophageal heat transfer device can also comprise a gastric access lumen. For example, the esophageal heat transfer device can comprise a gastric access tube defining the gastric access lumen.

Certain embodiments of the present invention can utilize a controller such as that described in US20070203552 (Machold). For example, a controller can employ a Peltier module to heat or cool a heat exchange region of the device, which would allow the device to alternate between heating and cooling modalities merely by changing the polarity of the current flowing through the module. In addition, the amount of heat or cold generated can be adjusted by controlling the amount of current flowing through the module. In particular, a controller can comprise a proportional-integral-derivative controller (PID controller) or a proportional controller.

In general, the controller can include a controlled variable, such as pump output or power input to the heat exchanger. A detecting unit or sensor can act as a feedback device for detecting a parameter, such as patient temperature or the presence of air in a line, and outputting a feedback signal relative to the control variable. The control unit can perform, for example, a PID operation, in which the controlled variable is adjusted according to the comparison between the feedback signal and a predetermined target value.

As used herein, the words “a,” “an,” and “the” mean “one or more,” unless otherwise specified. In addition, where aspects of the present technology are described with reference to lists of alternatives, the technology includes any individual member or subgroup of the list of alternatives and any combinations of one or more thereof.

The disclosures of all patents and publications, including published patent applications, are hereby incorporated by reference in their entireties to the same extent as if each patent and publication were specifically and individually incorporated by reference.

It is to be understood that the scope of the present invention is not to be limited to the specific embodiments described above. The present invention may be practiced other than as particularly described and still be within the scope of the accompanying claims.

Likewise, the following examples are presented in order to more fully illustrate the present invention. They should in no way be construed, however, as limiting the broad scope of the invention disclosed herein.

### EXAMPLE 1

### Operative Temperature Management in Pediatric-Sized Subjects

Three female Yorkshire swine (ranging from about 21 to about 26 kg; 22.7 kg±1.8 kg) were anesthetized with inhalational isoflurane via endotracheal intubation and instrumented.

A heat transfer device according to the present invention was inserted orally into the esophagus, with placement confirmed via auscultation and suction of gastric contents through a central suction channel. Temperature was monitored continuously via rectal thermistor placed after sedation for anesthesia and endotracheal intubation.

An external chiller (Gaymar MediTherm III) was utilized to provide a controlled temperature heat transfer medium to the heat transfer device. The specific heat transfer medium utilized was water. The device comprised lumens or channels through which the heat transfer medium flowed. Swine temperature, measured rectally, was reduced to, or maintained at, goal temperature by setting the chiller to automatic cooling mode.

The conditions for the experiments simulated an extremely challenging environment to maintain normothermia and/or re-warm a subject. The ambient temperature of the surgery suite measured about 70° F. (about 21° C.). The temperature of the air being delivered to the suite from the HVAC system was about 57° F. (about 14° C.). IV fluids were room temperature.

Subject #1 was not covered with any blankets. Subject #2 and Subject #3 were covered with blankets to minimize passive cooling due to the cold operating room conditions.

The average baseline temperature for the 3 animals was 38.3° C. (range 37.8° C. to 38.8° C.). Subject #1 experienced a maximum temperature decrease of 3.5° C./h and reached goal temperature in 83 min. Passive cooling contributed up to 1.8° C./h of this rate in Subject #1. Subject #2 and Subject #3 experienced maximum temperature decreases of 1.5° C./h and 1.7° C./h and reached goal temperature in 180 min and 182 min respectively. The actual heat reduction attributable to the device for the pediatric-sized subjects was about 1.7° C./h, which is comparable to the heat reduction observed in larger subjects. No treatment for shivering was necessary during the protocol. Upon inspection, esophageal gross pathology appeared unremarkable.

For Subject #3, the initial portion of the protocol was designed to maintain normothermia following the initial temperature drop caused by the anesthesia. Using the esophageal heat transfer device and blankets, without warmed IV fluids or any other active warming modality, normothermia was maintained in Subject #3. Thus, an exemplary esophageal heat transfer device of the present invention is capable of warming a pediatric-sized subject in an environment that is not conducive to the maintenance of normothermia. Indeed, in a cooling-biased environment, the exemplary esophageal heat transfer device halted the environmentally-induced decrease in core body temperature and stabilized the subject's body temperature.

Once Subject #3 had stabilized at normothermia, the external chiller was set to automatic cooling mode, with the objective of cooling the subject to goal temperature, without experiencing the substantial overshoot. Relying entirely on the chiller's internal PID controller algorithm, a cooling rate of 1.5° C./h was achieved before the chiller automatically began raising the coolant temperature to asymptotically approach the goal temperature, which was hit perfectly, and maintained exactly.

The esophageal heat transfer device successfully induced therapeutic hypothermia and maintained normothermia in the pediatric model. Maximum cooling rates exceeded expectations and goal temperature was attained faster than expected for all subjects, even with skin surface covering. Additionally, no thermogenic shivering occurred. Passive cooling was a significant factor for the uncovered subject, but the esophageal heat transfer device was still highly effective for covered subjects.

The foregoing description of the present invention provides illustration and description, but is not intended to be exhaustive or to limit the invention to the precise one disclosed. Modifications and variations are possible in light of the above teachings or may be acquired from practice of the invention. Thus, it is noted that the scope of the invention is defined by the claims and their equivalents.

What is claimed is:

1. A method for maintaining normothermia in a pediatric subject in a cooling-biased environment, comprising:
performing anesthesia on the pediatric subject in the cooling-biased environment;
orally or nasally inserting an esophageal heat transfer device into the pediatric subject, the esophageal heat transfer device including a heat transfer region and a gastric access tube;
positioning the heat transfer device such that at least a portion of the heat transfer region is in an esophagus of the pediatric subject and a distal end of the gastric access tube is in a stomach of the pediatric subject;
suctioning contents of the stomach of the pediatric subject via the gastric access tube of the heat transfer device;
contacting the heat transfer region with esophageal tissue of the esophagus of the pediatric subject; and
transferring heat between the esophageal heat transfer device and the esophageal tissue via the heat transfer region to facilitate normothermia of the pediatric subject.

2. The method of claim 1, further including covering the pediatric subject with a blanket to further facilitate normothermia of the pediatric subject.

3. The method of claim 2, further including pre-warming the blanket prior to covering the pediatric subject with the blanket to further facilitate normothermia of the pediatric subject.

4. The method of claim 1, wherein, to increase heat transfer between the heat transfer region and the esophageal tissue, the gastric access tube suctions the contents of the stomach before the heat transfer region contacts the esophageal tissue to improve contact between the heat transfer region and the esophageal tissue.

5. The method of claim 1, wherein the pediatric subject is less than about 26 kg.

6. The method of claim 1, wherein the cooling-biased environment includes a surgical suit with an ambient temperature of about 23° C. or less.

7. The method of claim 1, further including causing fluid to flow through a fluid path define by one or more lumens of the heat transfer device.

8. The method of claim 7, wherein flowing the fluid through the fluid path enables the transfer of heat between the heat transfer region and esophageal tissue.

9. The method of claim 8, further including controlling the fluid flowing through the fluid path via a PID controller to control the transfer of heat between the heat transfer region and esophageal tissue.

10. A method for temperature management of a pediatric subject, comprising:
inducing hypothermia of the pediatric subject experiencing hypoxic ischemic encephalopathy by:
orally or nasally inserting an esophageal heat transfer device into the pediatric subject, the esophageal heat transfer device including a heat transfer region;
positioning the heat transfer device such that at least a portion of the heat transfer region is in an esophagus of the pediatric subject;
contacting the heat transfer region with esophageal tissue of the esophagus of the pediatric subject; and
cooling a body of the pediatric subject by transferring heat from the esophageal tissue of the pediatric subject to the heat transfer region of the heat transfer device; and
after symptoms of the hypoxic ischemic encephalopathy are reduced, warming the body of the pediatric subject by transferring heat from the heat transfer region of the heat transfer device to the esophageal tissue of the pediatric subject.

11. The method of claim 10, further including covering the pediatric subject with a blanket after symptoms of the hypoxic ischemic encephalopathy are reduced to further warm the pediatric subject.

12. The method of claim 11, further including pre-warming the blanket prior to covering the pediatric subject with the blanket to further warm the pediatric subject.

13. The method of claim 10, further including, to increase heat transfer between the heat transfer region and the esophageal tissue, suctioning contents of a stomach of the pediatric subject via a gastric access tube of the heat transfer device to improve contact between the heat transfer region and the esophageal tissue.

14. The method of claim 10, wherein the pediatric subject is less than about 26 kg.

15. The method of claim 10, wherein hypothermia is induced in an environment having an ambient temperature of about 21° C. or less.

16. The method of claim 10, further including causing fluid to flow through a fluid path define by one or more lumens of the heat transfer device to transfer heat between the heat transfer region and esophageal tissue.

17. The method of claim 16, further including causing cool fluid to flow through the fluid path to induce hypothermia and subsequently causing warm fluid to flow through the fluid path to warm the pediatric patient.

18. The method of claim 17, further including controlling the fluid flowing through the fluid path via a PID controller to control the transfer of heat between the heat transfer region and esophageal tissue.

19. A method for temperature management of a pediatric subject, comprising:
inducing hypothermia of the pediatric subject experiencing cardiac arrest by:
orally or nasally inserting an esophageal heat transfer device into the pediatric subject, the esophageal heat transfer device including a heat transfer region;
positioning the heat transfer device such that at least a portion of the heat transfer region is in an esophagus of the pediatric subject;
contacting the heat transfer region with esophageal tissue of the esophagus of the pediatric subject; and
cooling a body of the pediatric subject by transferring heat from the esophageal tissue of the pediatric subject to the heat transfer region of the heat transfer device; and
after symptoms of the cardiac arrest are reduced, warming the body of the pediatric subject by transferring heat from the heat transfer region of the heat transfer device to the esophageal tissue of the pediatric subject.

20. The method of claim 19, further including covering the pediatric subject with a blanket after symptoms of the cardiac arrest are reduced to further warm the pediatric subject.
